SEQUENCE LISTING (6 sequences)

SEQ ID NO: 1 - RNA, 21 nt, Artificial Sequence
  sense (Antisense Bcl-2)
aagaagaggagaaaaaaauga

SEQ ID NO: 2 - RNA, 21 nt, Artificial Sequence
  antisense (Antisense BI-1)
ucauuucuucucuuucuucuu

SEQ ID NO: 3 - DNA, 21 bp, Artificial Sequence
  Antisense Bcl-2
aagaagaggagaaaaaaatga

SEQ ID NO: 4 - DNA, 21 bp, Artificial Sequence
  Antisense BI-1
tcatttcttctctttcttctt

SEQ ID NO: 5 - DNA, 54 bp, Artificial Sequence
  TTCAAGAGAG loop shRNA
aagaagaggagaaaaaaatgattcaagagagtcatttcttctctttcttctttt

SEQ ID NO: 6 - DNA, 54 bp, Artificial Sequence
  TTGGATCCAA loop shRNA
aagaagaggagaaaaaaatgattggatccaatcatttcttctctttcttctttt

(19) United States
(12) Patent Application Publication
(10) Pub. No.: US 2024/0153581 A1
(43) Pub. Date: May 9, 2024

(54) METHOD, DEVICE, COMPUTER PROGRAM AND COMPUTER-READABLE RECORDING MEDIUM FOR DESIGNING NUCLEIC ACID MOLECULES

(71) Applicant: CURIGIN CO.,LTD., Seoul (KR)

(72) Inventors: Jung-Ki YOO, Yangju-si (KR); Chung-Gab CHOI, Incheon (KR); Ki-Hwan UM, Ansan-si (KR); Eui-jin LEE, Gunpo-si (KR)

(73) Assignee: CURIGIN CO.,LTD., Seoul (KR)

(21) Appl. No.: 18/281,529

(22) PCT Filed: Nov. 19, 2020
(86) PCT No.: PCT/KR2020/016318

§ 371 Date: Sep. 11, 2023

(30) Foreign Application Priority Data
Dec. 5, 2019 (KR) 10-2019-0160720

(51) Int. Cl. G16B 25/30; G16B 30/10; G16B 30/20; G16B 35/20

(52) U.S. Cl. CPC G16B 25/30; G16B 30/10; G16B 30/20; G16B 35/20

(57) ABSTRACT

Disclosed is a method comprising extracting a gene sequence of a first gene from a first database in response to receiving a user's input, generating segmented sequences on the basis of the reverse complementary sequence of the gene sequence of the first gene, identifying, based on comparison of the segmented sequences with gene sequences of a second database, at least one matched sequence corresponding to at least one segmented sequence of the segmented sequences.

### CROSS REFERENCE TO RELATED APPLICATIONS

This application is a National Stage of International Application No. PCT/KR2020/016318 filed Nov. 19, 2020, claiming priority based on Korean Patent Application No. 10-2019-0160720 filed Dec. 5, 2019, the entire disclosures of which are incorporated herein by reference.

### INCORPORATION BY REFERENCE OF SEQUENCE LISTING

The content of the electronically submitted sequence listing, file name: Q286121 sequence listing as filed; size: 1,508 bytes; and date of creation: Sep. 11, 2023, filed herewith, is incorporated herein by reference in its entirety.

### TECHNICAL FIELD

The disclosed embodiments relate to designing nucleic acid molecules, and more particularly, to a method, a device, a computer program, and a computer readable medium for designing nucleic acid molecules. The disclosed embodiment relates to designing dual target nucleic acid molecules, and more specifically, to a method for designing dual target nucleic acid molecules, a device for designing the same, a computer program, and a recording medium recording the same.

### BACKGROUND ART

The technology of inhibiting gene expression is an important tool in the development of therapeutic agents and validation of targets for disease treatment. RNA interference (hereinafter referred to as ‘RNAi’) has been shown to act on sequence-specific mRNAs in various types of mammalian cells (Silence of the transcripts: RNA interference in medicine. J Mol Med (2005) 83: 764773) since its role was discovered. The RNAi is a phenomenon in which a small interfering ribonucleic acid (small interfering RNA, hereinafter referred to as ‘siRNA’) having a double helix structure of 21 to 25 nucleotides in size specifically binds to an mRNA transcript having a complementary sequence to decompose the mRNA transcript and inhibit expression of a specific protein. In cells, an RNA double strand is processed by an endonuclease, which is called Dicer, to be converted to an siRNA of a double strand of 21 to 23 base pairs (bps), and an siRNA binds to an RNA-induced silencing complex (RISC) so that a guide (antisense) strand sequence-specifically inhibits expression of a target gene through a process of recognizing and decomposing a target mRNA (NUCLEIC-ACID THERAPEUTICS: BASIC PRINCIPLES AND RECENT APPLICATIONS. Nature Reviews Drug Discovery. 2002. 1, 503-514). According to a Bertrand's research team, it was found that an siRNA for the same target gene had a superior inhibitory effect on mRNA expression in vitro and in vivo compared to antisense oligonucleotide (ASO) and the effect included a long-lasting effect (Comparison of antisense oligonucleotides and siRNAs in cell culture and in vivo. Biochem. Biophys. Res. Commun. 2002. 296: 1000-1004). A global market for therapeutics based on RNAi technology, including siRNA, is expected to reach more than 12 trillion won around 2020, and the number of targets to which these technologies can be applied is expected to expanded dramatically so these technologies are evaluated as a next-generation gene therapy technology capable of treating diseases that are hardly treated with existing antibody and compound-based medicines. In addition, since a mechanism of action of an siRNA is to complementarily bind to a target mRNA to regulate expression of a target gene in a sequence-specific manner, the number of applicable targets can be dramatically expanded, and while the development period is shortened, there is an advantage of being able to develop lead compounds optimized for all protein targets, including non-medicatable target substances compared to a long development period and development cost required for existing antibody-based medicines or small molecule drugs to be optimized for a specific protein target (Progress Towards in Vivo Use of siRNAs. MOLECULAR THERAPY. 2006 13(4):664-670). Therefore, recently, this ribonucleic acid-mediated interference phenomenon has presented a solution to a problem that occurs in the development of existing chemical synthetic medicines, and studies to selectively inhibit expression of specific proteins at a transcript level for the development of various disease therapeutic agents, especially tumor therapeutic agents are in progress. In addition, unlike conventional anticancer agents, siRNA therapeutics have an advantage of having a clear target and predictable side effects, but in the case of tumors, which are diseases caused by problems of various genes, such target specificity may rather cause a low therapeutic effect. In the case of tumors, controlling one gene does not cure a cancer, and resistance of anticancer drugs often develops. Accordingly, when treating a cancer with a gene therapy, it is difficult to control the cancer only by targeting one gene. Preparing siRNAs for several genes and introducing each of them is unlikely to achieves a desired effect for solving the difficulty due to a limit of the number to be delivered by a vector and an increase of an off-target effect. Therefore, it is necessary to design a sequence so that one nucleic acid sequence may target many genes related to a target disease at the same time, but it is very difficult to design dual target nucleic acid molecules due to the diversity of sequences and the possibility of off-target existence. Accordingly, the present disclosure intends to disclose a method for efficiently designing dual target nucleic acid molecules.

### DISCLOSURE

### Technical Problem

The present disclosure is directed to providing a method and a device for designing a sequence in which one strand of a double-stranded nucleic acid sequence targets a first gene and the other strand targets a second gene.

### Technical Solution

An exemplary embodiment of the present disclosure provides a method for designing a double nucleic acid molecule including: extracting a gene sequence of a first gene from a pre-formed gene database; segmenting the extracted gene sequence of the first gene sequentially from 5′ or 3′ end of the extracted gene sequence of the first gene with a size of 25 to 40 bp, wherein each segmented sequence has front and rear ends overlapped by 5 to 20 bp with segmented sequences located before and after the segmented sequence; aligning the segmented sequences with gene sequence of a pre-formed gene database; and scoring the sequences aligned with the segmented sequences in the pre-formed gene database.

In one exemplary embodiment of the present disclosure, the aligning step may employ a reverse complementary sequence of the segmented sequence.

In one exemplary embodiment of the present disclosure, the first gene targeted by the segmented sequence and a second gene targeted by the sequence aligned with the segmented sequence may be genes related to a same disease.

According to one exemplary embodiment of the present disclosure, in the scoring step, when the segmented sequence may be given with a weight in cases where 1) the sequence is not located within a distance of 75 bp from a start codon, 2) content of G bases and C bases is 36% to 52% with respect to the total number of bases, 3) a GC repeat sequence is less than 3, 4) an AT repeat sequence is less than 4, 5) a G base or a C base is present at the 1st position when the segmented sequence is a sense sequence, 6) an A base is present at the 3rd position when the segmented sequence is a sense sequence, 7) a T or U base is present at the 10th position when the segmented sequence is a sense sequence, 8) a G base is not present at the 13th position when the segmented sequence is a sense sequence, 9) an A base is present at the 19th position when the segmented sequence is a sense sequence, 10) a G base or C base is not present at the 19th position when the segmented sequence is a sense sequence, 11) an A base, T base, or U base is not present at the 1st position when the segmented sequence is an antisense sequence, 12) a G base is present at the 1st position of the sequence when the segmented sequence is an antisense sequence, 13) an A base is present at the 6th position when the segmented sequence is an antisense sequence, 14) a G base or C base is not present at the 19th position of the sequence when the segmented sequence is an antisense sequence, 15) a U base or T base is present at the 19th position of the sequence when the segmented sequence is an antisense sequence, and 16) the segmented sequence is located at a coding sequence (CDS).

According to one exemplary embodiment of the present disclosure, in the scoring step, the sequence matched and aligned with the segmented sequence may be given with a weight in cases where 1) the matched sequence is not located within a distance of 75 bp from a start codon, 2) content of G bases and C bases is 36% to 52% with respect to the total number of bases, 3) a GC repeat sequence is less than 3, 4) an AT repeat sequence is less than 4, 5) a G base or a C base is present at the 1st position when the matched sequence is a sense sequence, 6) an A base is present at the 3rd position when the matched sequence is a sense sequence, 7) a T or U base is present at the 10th position when the matched sequence is a sense sequence, 8) a G base is not present at the 13th position when the matched sequence is a sense sequence, 9) an A base is not present at the 19th position when the matched sequence is a sense sequence, 10) a G base or C base is not present at the 19th position when the matched sequence is a sense sequence, 11) an A base, T base, or U base is not present at the 1st position when the matched sequence is an antisense sequence, 12) a G base is present at the 1st position of the sequence when the matched sequence is an antisense sequence, 13) an A base is present at the 6th position when the matched sequence is an antisense sequence, 14) a G base or C base is not present at the 19th position of the sequence when the matched sequence is an antisense sequence, 15) a U base or T base is present at the 19th position of the sequence when the matched sequence is an antisense sequence, and 16) the matched sequence is located at a coding sequence (CDS).

According to an exemplary embodiment of the present disclosure, the scoring step may include: 1) applying a weight when a number of mismatches between the segmented sequence and the sequence matched with the segmented sequence is 5 or less.

According to an exemplary embodiment of the present disclosure, the nucleic acid molecule may be siRNA or shRNA, may be double-stranded, and may have a structure in which one strand forms a hairpin structure.

According to an exemplary embodiment of the present disclosure, the siRNA may consist of 19 to 24 bp.

According to an exemplary embodiment of the present disclosure, the alignment may be performed using a striped smith-waterman algorithm.

According to an exemplary embodiment of the present disclosure, after the scoring step, the method may include aligning the scores from a high rank to a low rank. Further, after the scoring step, the method may include aligning each of the segmented sequence and the sequence matched and aligned with the segmented sequence with sequences of a pre-formed database for gene transcript and then selecting, as a dual target nucleic acid molecule, when there is no gene transcript with which each of the segmented sequence and the sequence matched and aligned with the segmented sequence is matched except for self-derived gene transcripts. In addition, the selected dual target nucleic acid molecule may target a tumor suppressor gene.

### Advantageous Effects

According to the present disclosure, it is possible to efficiently design dual target nucleic acid molecules by using the method and the device.

### DESCRIPTION OF DRAWINGS

FIG. 1 is a flowchart of a method for designing a dual target nucleic acid molecule according to an exemplary embodiment.

FIG. 2 is a block diagram of a device for designing a dual target nucleic acid molecule according to an exemplary embodiment.

FIG. 3 is a flowchart of a method for designing a nucleic acid molecule according to an exemplary embodiment.

### MODES OF THE INVENTION

Terms used in the present specification will be described in brief and the present disclosure will be described in detail.

Terms used in the present disclosure adopt general terms which are currently widely used as possible by considering functions in the present disclosure, but the terms may be changed depending on an intention of those skilled in the art, a precedent, emergence of new technology, etc. Further, in a specific case, a term which an applicant arbitrarily selects is present and in this case, a meaning of the term will be disclosed in detail in a corresponding description part of the invention. Accordingly, a term used in the present disclosure should be defined based on not just a name of the term but a meaning of the term and contents throughout the present disclosure.

Throughout the specification, unless explicitly described to the contrary, when a part “includes” a certain component, it means that the part may further include other components without excluding other components. In addition, terms “unit’, “module”, and the like disclosed in the specification mean a unit that processes at least one function or operation and this may be implemented by hardware or software or a combination of hardware and software.

Hereinafter, the present disclosure will be described in detail with reference to the accompanying drawings.

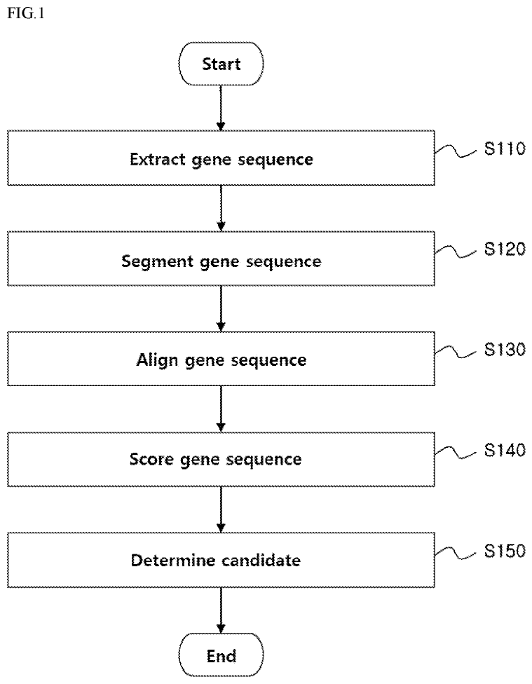

FIG. 1 is a flowchart of a method for designing a dual target nucleic acid molecule according to an exemplary embodiment.

In one exemplary embodiment, a device in which the method for designing the nucleic acid molecule is performed may be referred to as a device, an electronic device, a terminal, or the like. The device in which the method for designing the nucleic acid molecule is performed may be a computing device, but is not limited thereto, and may be a personal computer as well as a computer for designing nucleic acid molecules.

A gene sequence may be extracted in step S110.

The gene sequence may be extracted according to a user's selection from a pre-formed gene database, and the gene sequence may be a first gene sequence.

The first gene may be selected from genes known to cause a specific disease, but is not limited thereto. The first gene may be preferably selected from genes related to cancer. The device may include one or more processors. The deriving of the gene sequence may be a reverse complementary sequence to be described below. The pre-formed database may be a National Center for Biotechnology Information (NCBI) database, but is not limited thereto.

The gene sequence of the first gene extracted in step S120 may be segmented.

In one exemplary embodiment, sequence alignment may be performed after the reverse complementary sequence of the gene sequence of the first gene is segmented. In one exemplary embodiment, the gene sequence of the first gene may be segmented, and the sequence alignment may be performed on the reverse complementary sequence of the segmented gene sequence.

The gene sequence of the first gene may be segmented with a size of 25 to 40 base pairs (bp), preferably 30 bp, sequentially from the 5′ or 3′ end of the gene sequence of the first gene. In addition, each segmented sequence may be segmented so that its front and rear ends are overlapped by 5 to 20 bp, preferably 10 bp, with sequences before and after the segmented sequence. Here, a gene sequence usable as siRNA having a length of 19 to 24 bp may be designed by performing the segmentation with a size of 25 bp to 40 bp. Furthermore, since each segmented sequence is overlapped with other sequences ahead and behind, it is possible to prevent sequences from being omitted during subsequent alignment. In the case of segmenting the gene sequence in S120, the segmentation may be performed using the reverse complementary sequence of the first gene.

In the present disclosure, the reverse complementary sequence refers to a sequence that is in an opposite direction of a gene sequence indicated from 5′ to 3′ and is indicated from 5′ to 3′. More specifically, a general way of indicating a gene sequence is to indicate only one strand of a double strand of the gene in the 5′ to 3′ direction from the position of a promoter, and, when transcribing the gene indicated in the 5′ to 3′ direction, its complementary sequence that is a 3′ to 5′ sequence is used as a template. As a result, a 5′ to 3′ sequence in the direction of the promoter position corresponds to a sequence that is matched with the sequence of an mRNA transcript (a single strand, in the case of mRNA sequence). Here, the same sequence as mRNA is referred to as a sense sequence, and a sequence complementary to the sense sequence is referred to as an antisense sequence. Here, a sequence, indicated in the 5′ to 3′ direction, as a sequence of the strand used as the template is is referred to as a reverse complementary sequence. That is, a reverse complementary sequence of a gene sequence indicated in 5′ATGCATGC 3′ is 5′GCATGCAT3′. In addition, a sequence transcribed into mRNA of the gene sequence of 5′ATGCATGC 3′ is 5′AUGCAUGC3′, and its antisense sequence is 5′GCAUGCAU 3′ or 5′GCATGCAT 3′.

A matched gene sequence may be identified in the pre-formed gene database by aligning the segmented sequences with gene sequences of the pre-formed gene database, database in step S130.

The alignment in the present disclosure may refer to a process of comparing gene sequences in life science. A reference sequence that is a gene sequence similar to the segmented sequences may be identified upon the alignment according to the present disclosure. For example, reference sequences that are genes or base sequences having sequence similarity to the segmented sequences may be identified among the gene sequences stored in the pre-formed gene database. The identified sequences may be listed based on similarity to the reference sequence. Here, the sequences that are identified or listed based on similarity are referred to as “matched sequences” in the present disclosure. In the present disclosure, the “matched sequence” may be used as a concept including not only a sequence in the 5′ to 3′ direction of the matched sequence but also a reverse complementary sequence in an opposite direction. That is, if the segmented sequence is 5′ATGCATGC 3′ (hereinafter, the antisense sequence for the first gene), its matched sequence may be 5′ATGCATGC 3′ (not necessarily 100% complementary), and its reverse complementary sequence, 5′GCATGCAT3′ sequence also may be referred to as the matched sequence. Here, when the reverse complementary sequence of the segmented sequence is used as an antisense targeting the first gene, a 5′ to 3′ sequence of the matched sequence may be used as an antisense targeting a second gene, and when the segmented sequence itself is used as the antisense targeting the first gene, the reverse complementary sequence of the matched sequence may be used as the antisense targeting the second gene.

In the present disclosure, during alignment, a striped smith-waterman algorithm, a needleman-wunsch algorithm, a levenshtein distance algorithm, a heuristic algorithm, or a hamming distance algorithm may be used, and preferably a striped smith-waterman algorithm may be used, but is not limited thereto. In addition, in step S130, if a gene (hereinafter referred to as the second gene) from which the matched sequences are derived is related to the same disease as the first gene, only the matched sequences may be selected for performing subsequent steps. That is, in step S130, an object to be compared with the segmented sequence may be a gene sequence of a gene related to the same disease as the first gene from which the segmented gene sequence is derived.

In step S140, scoring of sequences matched with the segmented sequences may be performed. The performance of scoring may be referred to as scoring. In one exemplary embodiment, pairs of the segmented sequences and the matched sequences may be scored. In one exemplary embodiment, weights may be applied based on sequence characteristics of the segmented sequences and the matched sequences. In one exemplary embodiment, the weights may be applied to pairs of the segmented sequences and the matched sequences. In one exemplary embodiment, a first weight may be applied to a gene sequence having a first sequence characteristic, and a second weight, greater than the first weight, may be applied to a gene sequence having a second sequence characteristic. The second weight may be twice greater than the first weight, but is not limited thereto. For example, the first weight may be 1 and the second weight may be 2.

In one exemplary embodiment, the weight may be applied to the segmented sequence according to the presence or absence of a specific base at a specific position of the segmented sequence.

In one exemplary embodiment, the weight may be applied to the segmented sequence according to a position of the first gene of the segmented sequence.

In one exemplary embodiment, the weight may be applied to the segmented sequence according to the number of repetitions of a specific base sequence in the segmented sequence.

In one exemplary embodiment, the weight may be applied to the segmented sequence according to content of specific bases in the segmented sequence.

In one exemplary embodiment, the weight may be applied to the segmented sequence according to the presence or absence of asymmetric base pairing in the segmented sequence.

In one exemplary embodiment, the weight may be applied to the segmented sequence according to a position of the energy valley in the segmented sequence.

In one exemplary embodiment, the weight may be applied to the segmented sequence according to the presence or absence of a specific structure in the segmented sequence.

The weight may be applied to the segmented sequence, in cases where 1) the segmented sequence is not located within a distance of 75 bp from a start codon, 2) content of G bases and C bases of the segmented sequence is 36% to 52% with respect to the total number of bases of the segmented sequence, 3) a GC repeat sequence in the segmented sequence is less than 3, 4) an AT repeat sequence in the segmented sequence is less than 4, 5) a G base or a C base is present at the 1st position of the sequence when the segmented sequence is a sense sequence, 6) an A base is present at the 3rd position of the sequence when the segmented sequence is a sense sequence, 7) a T or U base is present at the 10th position of the sequence when the segmented sequence is a sense sequence, 8) a G base is not present at the 13th position of the sequence when the segmented sequence is a sense sequence, 9) an A base is present at the 19th position of the sequence when the segmented sequence is a sense sequence, 10) a G base or C base is not present at the 19th position of the sequence when the segmented sequence is a sense sequence, 11) an A base, T base, or U base is not present at the 1st position of the sequence when the segmented sequence is an antisense sequence, 12) a G base is present at the 1st position of the sequence when the segmented sequence is an antisense sequence, 13) an A base is present at the 6th position of the sequence when the segmented sequence is an antisense sequence, 15) a G base or C base is not present at the 19th position of the sequence when the segmented sequence is an antisense sequence, 15) a U base or T base is present at the 19th position of the sequence when the segmented sequence is an antisense sequence, 16) the segmented sequence is located at a coding sequence (CDS), 17) the segmented sequence is not located at single nucleotide polymorphism (SNP) positions, 18) content of G bases and C bases of the sequence from the 2nd position to the 7th position is 19% of the total G bases and C bases, and content of G bases and C bases of the sequence from the 8th position to the 18th position is 52% of the total G bases and C bases when the segmented sequence is an antisense sequence, 19) there is asymmetrical base pairing in a duplex of the segmented sequence, 20) there is an energy valley in the sequence from the 19th position to the 14th position of the sequence when the segmented sequence is a sense sequence, and 21) the segmented sequence has no internal secondary structure and no hairpins.

In one exemplary embodiment, the weight may be applied to the matched sequence according to the presence or absence of a specific base at a specific position of the matched sequence.

In one exemplary embodiment, the weight may be applied to the matched sequence according to a position of the second gene of the matched sequence.

In one exemplary embodiment, the weight may be applied to the matched sequence according to the number of repetitions of a specific base sequence in the matched sequence.

In one exemplary embodiment, the weight may be applied to the matched sequence according to content of specific bases in the matched sequence.

In one exemplary embodiment, the weight may be applied to the matched sequence according to the presence or absence of asymmetric base pairing in the matched sequence.

In one exemplary embodiment, the weight may be applied to the matched sequence according to a position of the energy valley in the matched sequence.

In one exemplary embodiment, the weight may be applied to the matched sequence according to the presence or absence of a specific structure in the matched sequence.

The weight may be applied to the matched sequence, in cases where 1) the matched sequence is not located within a distance of 75 bp from a start codon, 2) content of G bases and C bases of the matched sequence is 36% to 52% with respect to the total number of bases of the matched sequence, 3) a GC repeat sequence in the matched sequence is less than 3, 4) an AT repeat sequence in the matched sequence is less than 4, 5) a G base or a C base is present at the 1st position of the sequence when the matched sequence is a sense sequence, 6) an A base is present at the 3rd position of the sequence when the matched sequence is a sense sequence, 7) a T or U base is present at the 10th position of the sequence when the matched sequence is a sense sequence, 8) a G base is not present at the 13th position of the sequence when the matched sequence is a sense sequence, 9) an A base is present at the 19th position of the sequence when the matched sequence is a sense sequence, 10) a G base or C base is not present at the 19th position of the sequence when the matched sequence is a sense sequence, 11) an A base, T base, or U base is not present at position 1 of the sequence when the matched sequence is an antisense sequence, 12) a G base is present at the 1st position of the sequence when the matched sequence is an antisense sequence, 13) an A base is present at the 6th position of the sequence when the matched sequence is an antisense sequence, 14) a G base or C base is not present at the 19th position of the sequence when the matched sequence is an antisense sequence, 15) a U base or T base is present at the 19th position of the sequence when the matched sequence is an antisense sequence, 16) the matched sequence is located at a coding sequence (CDS), 17) the matched sequence is not located at single nucleotide polymorphism (SNP) positions, 18) content of G bases and C bases of the sequence from the 2nd position to the 7th position is 19% of the total G bases and C bases, and content of G bases and C bases of the sequence from the 8th position to the 18th position is 52% of the total G bases and C bases when the matched sequence is an antisense sequence, 19) there is asymmetrical base pairing in a duplex of the matched sequence, 20) there is an energy valley in the sequence from the 9th position to the 14th position of the sequence when the matched sequence is a sense sequence, and 21) the matched sequence has no internal secondary structure and no hairpins.

In addition, when the number of mismatches between the segmented sequence and the matched sequence is 5 or less, a weight may be applied to the segmented sequence and the matched sequence.

In the present disclosure, the “sense sequence” may mean a sequence which is same as 5′ to 3′ sequence of a gene to be targeted, that is, a sequence which is same as the mRNA expressed in the gene to be targeted, and the “antisense sequence” may mean a 3′ to 5′ sequence complementary to mRNA expressed from the gene to be targeted.

In addition, in the present disclosure, the coding sequence (CDS) may mean a sequence that is translated into a protein.

In addition, in the present disclosure, the start codon means a start site where mRNA of the gene, from which sequences are derived, is transcribed. For example, a requirement that sequences are not located in 75 bp from the start codon means a case where the segmented sequence is not derived from a sequence located within 75 bp from the start codon of the first gene from which the segmented sequence is derived.

In one exemplary embodiment, the weight is applied to each of the segmented sequence and the matched sequence, so that the segmented sequence and the matched sequence may be sorted in order of higher score.

In S150, a gene sequence candidate for targeting the second gene may be determined according to scores of the segmented sequence and the matched sequence.

Step S150 may include aligning each of the segmented sequence and the matched sequence with the sequences of a pre-formed database of gene transcripts.

Step S150 may be performed before or after step S140. In step S150, when the segmented sequence has no matched sequence except for the transcript of the first gene (the gene from which the segmented sequence is derived), and the aligned and matched sequence has no matched sequence except for the transcript of the second gene (the gene from which the aligned and matched sequence is derived), the segmented sequence and the matched sequence may be selected as dual target nucleic acid molecules.

During the alignment in step S150, a sequence in the 5′ to 3′ direction (the antisense sequence for the first gene) of the segmented sequence is used, and a 5′ to 3′ sequence (the antisense for the second gene) of the “matched sequence” may be used. More specifically, when the segmented sequence is 5′ATGCTAC 3′ and the matched sequence is 5′GTAGCAT3′, the segmented sequence 5′ATGCTAC 3′ targets the first gene and the matched sequence 5′GTAGCAT3′ targets the second gene. During the alignment in step S150, when the segmented sequence 5′ATGCTAC3′ is used and the matched sequence 5′GTAGCAT3′ is used, and there is no aligned and matched sequence except for the first gene and the second gene, then the segmented sequence 5′ATGCTAC3′ and the matched sequence 5′GTAGCAT3′ may be selected as dual target nucleic acid molecules.

In one exemplary embodiment, sequences that do not target a tumor suppressor gene among the segmented sequences and the matched sequences may be selected as dual target nucleic acid molecules. That is, when either the segmented sequence or the matched sequence matches the tumor suppressor gene, the matched sequence may be excluded from the candidates of dual target nucleic acid molecules. Thus, it is possible to prevent an off-target effect in which each of the segmented sequence and the “matched sequence” targets a gene other than the target gene to be targeted. The term “off-target effect” of the present disclosure refers to an effect that targets a gene other than the desired target gene.

In the present disclosure, the dual target nucleic acid molecule may be designed as siRNA or shRNA, but is not limited thereto. When designed as the siRNA, the dual target nucleic acid molecule may be double-stranded siRNA of 19 to 24 bp, in which one strand may be derived from the “segmented sequence”, that is, the first gene, and the other strand may be derived from the “matched sequence”, that is, the second gene. When designed as the shRNA, the dual target nucleic acid molecule may be designed as a structure including a sequence derived from the first gene, a structure that can form a hairpin structure, and a sequence derived from the second gene.

In the present disclosure, the targeting of the first gene or the second gene may mean targeting a transcript of the first gene or a transcript of the second gene.

The nucleic acid molecule of the present disclosure is for the purpose of targeting different genes, and the first gene targeted by the segmented sequence and the second gene targeted by the “matched sequence” may be genes related to the same disease, for example, cancer, that is, oncogenes, but are not limited thereto.

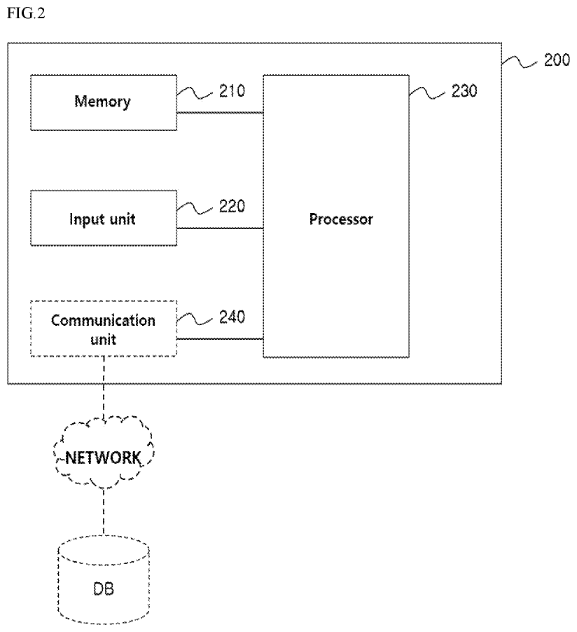

FIG. 2 is a block diagram of a device 200 of designing a dual target nucleic acid molecule according to an exemplary embodiment.

Referring to FIG. 2, the device 200 may include a memory 210, an input unit 220 and at least one processor 230. According to the method for designing the nucleic acid molecule proposed in the exemplary embodiments, the memory 210, the input unit 220, and the at least one processor 230 may operate. However, components of the device 200 according to an exemplary embodiment are not limited to the above-described example. According to another exemplary embodiment, the device 200 for designing the nucleic acid molecule may include more or fewer components than the aforementioned components. For example, the device 200 may further include a communication unit 240. The processor 230 may access an external database through the communication unit 240.

The memory 210 according to an exemplary embodiment may store a database which is pre-created based on gene sequence information. The database may be stored in the memory 210 of the device 200, but is not limited thereto. For example, the database may be located outside the device 200, and the device 200 may access the external database through the communication unit 240. The database may contain data from NCBI. In addition, according to another exemplary embodiment, the database may include genetic information obtained through NCBI.

A target gene name, sequence information, or the like may be input to the processor 230 from the user through the input unit 220 according to an exemplary embodiment. However, this is only an embodiment, and all user inputs required for designing a dual target nucleic acid sequence may be received by the processor 230 in various forms.

The at least one processor 230 according to an exemplary embodiment may segment a reverse complementary sequence of a first gene sequence using the pre-created database, and align the segmented sequences with gene sequences of the pre-created database, and score the matched sequences and select sequences with high scores according to specific criteria to determine candidate substances for the dual target nucleic acid molecule.

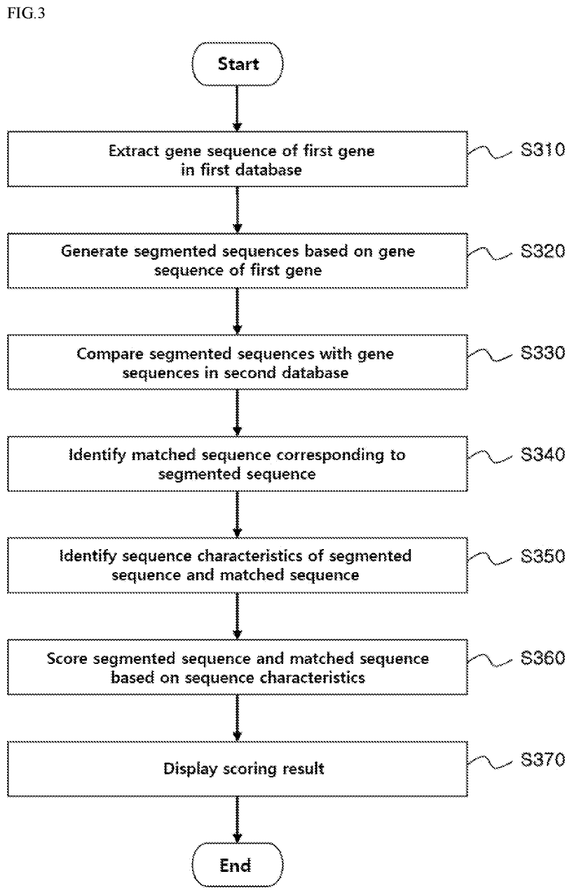

FIG. 3 is a flowchart of a method for designing a nucleic acid molecule according to an exemplary embodiment.

The method for designing the nucleic acid molecule of FIG. 3 may be performed by an electronic device, but is not limited thereto. For example, the method may be performed by the device 200 of FIG. 2.

Referring to FIG. 3, a gene sequence of a first gene may be extracted from a first database in S310. Since S310 is substantially the same as the aforementioned S110, the duplicated description will be omitted.

In one exemplary embodiment, the first database may be an NCBI database, but is not limited thereto.

In S320, segmented sequences may be generated based on the gene sequence of the first gene. In one exemplary embodiment, segmented sequences may be generated from a reverse complementary sequence of the first gene sequence. Both ends of each of the segmented sequences may overlap with both ends of other segmented sequences ahead and behind. Since S320 is substantially the same as the aforementioned S120, the duplicated description will be omitted.

In S330, the segmented sequences may be compared with gene sequences in a second database. The second database may be the same NCBI database as the first database, but is not limited thereto. Since S330 is substantially the same as some operations of the aforementioned S130, the duplicated description will be omitted.

In S340, matched sequences corresponding to the segmented sequences may be identified. Since S340 is substantially the same as some operations of the aforementioned S130, the duplicated description will be omitted.

In S350, sequence characteristics of the segmented sequence and the matched sequence may be identified. Since S350 is substantially the same as some operations of the aforementioned S130, the duplicated description will be omitted.

In S360, based on the identified sequence characteristics, scoring may be performed for the segmented sequence and the matched sequence. Since S360 is substantially the same as some operations of the aforementioned S140, the duplicated description will be omitted.

Values described as the sequence characteristics in the present disclosure may vary depending on a length of each of the segmented sequences or an overlapping length of the segmented sequences.

In S370, the scoring results may be displayed. Since S370 is substantially the same as some operations of the aforementioned S150, the duplicated description will be omitted. The device according to the present disclosure may include a processor, a memory storing and executing program data, a permanent storage such as a disk drive, a communication port communicating with an external device, a user interface device such as a touch panel, a key, and a button, and the like. Methods implemented by software modules or algorithms may be stored on a computer readable recording medium as computer-readable codes or program instructions that may be executed on the processor. Here, the computer readable recording medium includes magnetic storage media (e.g., read-only memory (ROM), random-access memory (RAM), floppy disk, hard disk, etc.), optical reading media (e.g., CD-ROM, and digital versatile disc (DVD)), and the like. The computer readable recording media may be stored and executed with codes which may be distributed in computer systems connected via a network and readable by a computer in a distribution method. The media are readable by a computer, stored in the memory, and may be executed in the processor.

All documents including publications, patent applications, patents, etc. cited in the present disclosure are illustrated by combining each cited reference individually and specifically, or combined with the present disclosure in the same manner as those combined and indicated in the present disclosure as a whole.

In order to understand the present disclosure, reference numerals are given in the preferred exemplary embodiments shown in the drawings, specific terms have been used to describe the exemplary embodiments of the present disclosure, but the present disclosure is not limited by the specific terms, and the present disclosure may include all components commonly conceived by those skilled in the art.

The present disclosure may be represented by functional block configurations and various processing steps. These functional blocks may be implemented as various numbers of hardware or/and software configurations for executing specific functions. For example, the present disclosure may adopt IC configurations, such as a memory, a processing, a logic, a look-up table, and the like, which may execute various functions by control of one or more microprocessors or other control devices. The components of the present disclosure include various algorithms implemented in combination of a data structure, processes, routines, or other programming configurations like being executed by software programming or software elements to be implemented by a programming or scripting language such as C, C++, Java, assembler, R, Python, and the like. Functional aspects may be implemented as an algorithm executed in one or more processors. In addition, the present disclosure may adopt the related art for electronic environment configuration, signal processing, and/or data processing. The terms “mechanism”, “element”, “means”, and “configuration” may be widely used and are not limited to mechanical and physical configurations. The terms may include the meaning of a series of processes (routines) of software in conjunction with a processor or the like.

The specific implementations described in the present disclosure are exemplary embodiments, and do not limit the scope of the present disclosure in any way. For brevity of the specification, descriptions of conventional electronic configurations, control systems, software, and other functional aspects of the systems may be omitted. In addition, the connection or connection members of lines between the components illustrated in the drawings exemplarily represent functional connections and/or physical or circuit connections, and may be illustrated as various functional connections, physical connections, or circuit connections that may be replaced or added in an actual device. In addition, unless specifically stated, such as “essential” or “important”, components may not necessarily be required for the application of the present disclosure.

In the specification of the present disclosure (especially, in appended claims), the use of the term “the” and similar instruction terms thereto may correspond to both singular and plural references. In addition, in the present disclosure, when the range is described, the disclosure applied with individual values belonging to the above range is included (unless expressly indicated otherwise) and therefore, each individual value configuring the range will be disclosed in the detailed description of the disclosure. Finally, unless the order is explicitly stated or stated to the contrary for steps configuring the method according to the present disclosure, the steps may be performed in any suitable order. The present disclosure is not necessarily limited to the order of description of the steps. All examples described herein or the terms indicative thereof (“for example”, etc.) used herein are merely to describe the present disclosure in more detail. Therefore, the scope of the present disclosure is not limited to the embodiments or exemplary terms unless limited by the appended claims. In addition, it should be apparent to those skilled in the art that various modifications, combinations, and alternations may be configured depending on design conditions and factors within the scope of the appended claims or equivalents thereof.

### Embodiment 1. Design of Dual Nucleic Acid Molecules

A gene sequence of cancer-inducing B-cell lymphoma 2 (BCL2) was selected as a first gene, and a reverse complementary sequence of BCL2 was extracted from NCBI. Thereafter, the reverse complementary sequence of BCL2 was segmented with 30 bp to overlap each other by 10 bp in the 5′ to 3′ direction. Thereafter, for each of the segmented sequences, alignment was performed with about 37961 gene sequences provided by NCBI using a Striped Smith-Waterman algorithm. Thereafter, the segmented sequence in BCL2 was given with a weight of 1 point in cases where 1) the sequence was not located within a distance of 75 bp from a start codon, 2) content of G bases and C bases was 36% to 52% with respect to the total number of bases, 3) a GC repeat sequence was less than 3, 4) an AT repeat sequence was less than 4, 5) a G base or a C base was present at the 1st position when the segmented sequence was a sense sequence, 6) an A base was present at the 3rd position when the segmented sequence was a sense sequence, 7) a T or U base was present at the 10th position when the segmented sequence was a sense sequence, 8) a G base was not present at the 13th position when the segmented sequence was a sense sequence, 9) an A base was present at the 19th position when the segmented sequence was a sense sequence, 10) a G base or C base was not present at the 19th position when the segmented sequence was a sense sequence, 11) an A base, T base, or U base was not present at position 1 when the segmented sequence was an antisense sequence, 12) a G base was present at the 1st position of the sequence when the segmented sequence was an antisense sequence, 13) an A base was present at position 6 when the segmented sequence was an antisense sequence, 15) a G base or C base was not present at the 19th position of the sequence when the segmented sequence was an antisense sequence, 15) a U base or T base was present at the 19th position of the sequence when the segmented sequence was an antisense sequence, and 16) the segmented sequence was located at a coding sequence (CDS).

The aligned and matched segment was given with a weight of 1 point in cases where 1) the sequence was not located within a distance of 75 bp from a start codon of a targeting gene (BI-1), 2) content of G bases and C bases was 36% to 52% with respect to the total number of bases, 3) a GC repeat sequence was less than 3, 4) an AT repeat sequence was less than 4, 5) a G base or a C base was present at the 1st position when the matched sequence is a sense sequence, 6) an A base was present at the 3rd position when the matched sequence was a sense sequence, 7) a T or U base was present at the 10th position when the matched sequence was a sense sequence, 8) a G base was not present at the 13th position when the matched sequence was a sense sequence, 9) an A base was present at the 19th position when the matched sequence was a sense sequence, 10) a G base or C base was not present at the 19th position when the matched sequence was a sense sequence, 11) an A base, T base, or U base was not present at position 1 when the matched sequence was an antisense sequence, 12) a G base was present at the 1st position of the sequence when the matched sequence was an antisense sequence, 13) an A base was present at the 6th position when the matched sequence was an antisense sequence, 14) a G base or C base was not present at the 19th position of the sequence when the matched sequence was an antisense sequence, 15) a U base or T base was present at the 19th position of the sequence when the matched sequence was an anti sense sequence, and 16) when the matched sequence was located at a coding sequence (CDS). When the number of mismatches between the segmented sequence and the sequence aligned and matched with the segmented sequence was 5 or less, a weight of 1 point was given, and sequences with the highest score were derived.

As the derived result, the aligned and matched sequence was a sequence derived from BI-1, and since BI-1 and BCL-2 were genes used as targets for cancer treatment, which was the same disease, so they were determined as dual target nucleic acid molecules. The determined nucleic acid molecules were designed as siRNA, and sequences in Table 1 below were obtained. In addition, each sequence of Table 1 was aligned once again to confirm matching with other genes, and to confirm that there was no matching.

TABLE 1

| | | SEQ |
|---|---|---|
| | Sequence | ID |
| siRNA | (5′ → 3′) | NO: |
| sense (Antisense | AAG AAG AGG AGA | 1 |
| Bcl-2) | AAA AAA UGA | |
| antisense | UCA UUU CUU CUC | 2 |
| (Antisense BI-1) | UUU CUU CUU | |

In siRNA of 21 mer consisting of SEQ ID NOs: 1 and 2, it was confirmed through a subsequent experiment that 15 mers of 21 mers were complementary to each other, siRNA (Antisense Bcl-2) of SEQ ID NO: 1 was complementarily bound to mRNA of Bcl-2, and siRNA (Antisense Bcl-1) of SEQ ID NO: 2 was complementarily bound to mRNA of BI-1 to simultaneously reduce the expression of the Bcl-2 and BI-1 genes.

### Embodiment 2. Design of Dual Target shRNA

In order to be able to express the dual target siRNA, designed in Example 1, in cells, shRNAs (TTCAAGAGAG loop shRNA and TTGGATCCAA loop shRNA) containing a DNA conversion sequence (SEQ ID NOS: 3 and 4) and a loop sequence of the siRNA double strand were prepared (Table 2). The prepared shRNAs were disposed after a U7 promoter (SEQ ID NO: 7) at cleavage positions of restriction enzymes PstI and EcoRV of a pE3.1 vector (FIG. 1), respectively, to construct a recombinant expression vector capable of expressing two types of shRNAs in cells, including dual target siRNAs targeting BCL2 and BI-1. As a result of examining whether Bcl-2 and BI-1 were simultaneously targeted using the prepared recombinant expression vector, an effect of simultaneously inhibiting BCL-2 and BI-1 was confirmed.

TABLE 2

| | | SEQ |
|---|---|---|
| | Sequence | ID |
| | (5′ → 3′) | NO: |
| Antisense | aagaagaggagaaaaaaatga | 3 |
| Bcl-2 | | |
| Antisens BI-1 | tcatttcttctctttcttctt | 4 |
| TTCAAGAGAG | aagaagaggagaaaaaaatga | 5 |
| loop shRNA | TTCAAGAGAGtcatttcttct | |
| | ctttcttcttTT | |
| TTGGATCCAA | aagaagaggagaaaaaaatga | 6 |
| loop shRNA | TTGGATCCAAtcatttcttct | |
| | ctttcttcttTT | |

1. A method performed by a computing device, comprising:
extracting a gene sequence of a first gene from a first database in response to receiving a user's input;
generating segmented sequences based on a reverse complementary sequence of the gene sequence of the first gene;
identifying, based on comparison of the segmented sequences with gene sequences of a second database, at least one matched sequence corresponding to at least one segmented sequence of the segmented sequences, wherein the at least one matched sequence targets a second gene different from the first gene;
identifying the sequence characteristics of the at least one segmented sequence and the at least one matched sequence;
scoring the at least one segmented sequence and the at least one matched sequence by applying a first weight to a first sequence characteristic among the sequence characteristics and applying a second weight, greater than the first weight to a second sequence characteristic among the sequence characteristics; and
displaying the at least one segmented sequence and the at least one matched sequence based on scores of the at least one segmented sequence and the at least one matched sequence.

2. The method of claim 1, wherein the first gene and the second gene are related to a same disease.

3. The method of claim 1, wherein the first sequence characteristic is related to at least one of:
presence and absence of a specific base at a specific position in the at least one segmented sequence and the at least one matched sequence;
a position of the at least one segmented sequence in the first gene, and a position of the at least one matched sequence in the second gene;
a number of repetitions of a specific base sequence in the at least one segmented sequence and the at least one matched sequence; and
a content of specific bases in the at least one segmented sequence and the at least one matched sequence.

4. The method of claim 1, wherein the second sequence characteristic is related to at least one of:
asymmetrical base pairing in the at least one segmented sequence and the at least one matched sequence;
a position of an energy valley in the at least one segmented sequence and the at least one matched sequence; and
absence of a specific structure in the at least one segmented sequence and the at least one matched sequence.

5. The method of claim 1, wherein a third weight is applied to the at least one segmented sequence and the at least one matched sequence in which a number of mismatches between the at least one segmented sequence and the at least one matched sequence is less than or equal to a predetermined value.

6. The method of claim 1, further comprising:
comparing the at least one segmented sequence and the at least one matched sequence with gene sequences in a third database.

7. The method of claim 6, wherein the comparing with the gene sequences of the third database comprises:
identifying a gene sequence matched with the at least one segmented sequence among the gene sequences of the third database; and
identifying a gene sequence matched with the at least one matched sequence among the gene sequences of the third database, and
further comprises selecting, as a dual target nucleic acid molecule, the at least one segmented sequence and the at least one matched sequence when the at least one segmented sequence is only matched with a transcript of the first gene and the at least one matched sequence is only matched with a transcript of the second gene.

8. The method of claim 7, wherein the determining of the dual target nucleic acid molecule comprises selecting, as the dual target nucleic acid molecule, the at least one segmented sequence and the at least one matched sequence which do not target a tumor suppressor gene.

9. A computer-readable recording medium recording a program for executing the method of claim 1 on a computer.

10. An electronic device comprising:
a memory storing at least one instruction; and
a processor configured to execute the at least one instruction to perform the steps of:
extracting a gene sequence of a first gene from a first database in response to receiving a user's input;
generating segmented sequences based on a reverse complementary sequence of the gene sequence of the first gene;
identifying, based on comparison of the segmented sequences with gene sequences of a second database, at least one matched sequence corresponding to at least one segmented sequence of the segmented sequences, wherein the at least one matched sequence targets a second gene different from the first gene;
identifying sequence characteristics of the at least one segmented sequence and the at least one matched sequence;
scoring the at least one segmented sequence and the at least one matched sequence by applying a first weight to a first sequence characteristic among the sequence characteristics and applying a second weight, greater than the first weight to a second sequence characteristic among the sequence characteristics; and
displaying the at least one segmented sequence and the at least one matched sequence based on scores of the at least one segmented sequence and the at least one matched sequence.
